(19) United States
(12) Patent Application Publication
(10) Pub. No.: US 2018/0008618 A1
(43) Pub. Date: Jan. 11, 2018

(54) HEALTH CARE

(71) Applicants: Anthony I. Rozmanith, Winchester, MA (US); Jolan S. Rozmanith, Winchester, MA (US)

(72) Inventors: Anthony I. Rozmanith, Winchester, MA (US); Jolan S. Rozmanith, Winchester, MA (US)

(21) Appl. No.: 15/644,276

(22) Filed: Jul. 7, 2017

Related U.S. Application Data
(60) Provisional application No. 62/359,889, filed on Jul. 8, 2016.

(51) Int. Cl. A61K 31/60; A61K 31/18; A61K 31/167; A61K 31/155; A61K 9/20; A61K 31/192; A61K 9/00

(52) U.S. Cl. CPC A61K 31/60; A61K 31/192; A61K 31/18; A61K 9/0053; A61K 31/155; A61K 9/20; A61K 31/167

(57) ABSTRACT

The treatment of Type 2 diabetes involving the use of a regimen comprising: (A) one or more glucose-lowering drugs (G-LDs) which function to lower the amount of glucose in the blood, preferably glyburide and metformin; (B) at least one non-steroidal anti-inflammatory drug (NSAID) selected from the group consisting of (1) magnesium salicylate tetrahydrate (hereafter “Mg salicylate”); (2) naproxen; and (3) ibuprofen; and (C) acetaminophen. Also, a regimen for use in deterring the development in an individual of a cardiovascular disease or of Alzheimer's and/or for treating individuals who are affected with such diseases, the regimen comprising aforementioned ingredients (B) and (C) above.

### CROSS REFERENCE TO RELATED APPLICATION

The present application claims the priority of U.S. Provisional Application No. 62/359,889, filed Jul. 8, 2017, which is incorporated herein by reference.

### SUMMARY OF THE INVENTION

One aspect of the present invention provides a method for treating an individual who has Type 2 diabetes comprising the practice by the individual of a regimen which includes the ingestion daily by the individual of:

(A) one or more glucose-lowering drugs (G-LDs) which function to lower the amount of glucose in the blood, preferably glyburide and metformin;

(B) at least one non-steroidal anti-inflammatory drug (NSAID) selected from the group consisting of (1) magnesium salicylate tetrahydrate (hereafter “Mg salicylate”); (2) naproxen; and (3) ibuprofen; and

(C) acetaminophen.

The use of the above regimen can be used to prevent, or to eliminate the severity of, or to delay the onset of Type 2 diabetes in an individual.

The total amount of regimen ingested on a daily basis by the individual is a pharmaceutically effective amount, as explained below.

In preferred form, the individual optionally lives a life style which reduces the severity of undesirable symptoms associated with Type 2 diabetes.

Examples of embodiments of the regiment and other aspects of the invention are described hereinafter.

### DETAILED DESCRIPTION OF THE INVENTION

In addition to treating individuals who suffer from Type 2 diabetes, the regimen of the present invention can be used to treat an individual to reduce the risk of the development of a cardiovascular disease or the development of Alzheimer's disease or to treat an individual that has a cardiovascular disease or Alzheimer's disease.

The term “a cardiovascular disease” (also referred to herein as “CVD” for convenience) is used in the broad sense to include a disease which affects adversely the heart or a blood vessel, that is, heart disease (known also as “cardiac disease”) or a disease of one or more blood vessels (known also as “vascular disease”).

Examples of heart disease include: angina; arrhythmia; congenital heart disease; coronary artery disease (CAD); dilated cardiomyopathy; heart attack (myocardial infarction); heart failure; hypertrophic cardiomyopathy; mitral regurgitation; mitral valve prolapse; and pulmonary stenosis.

Examples of vascular disease include carotid artery disease (stroke precursor), peripheral artery disease, and any other condition that results in the blockage of fresh (that is, oxygenated) blood supply to the heart or brain.

With regard to CVD, various types of individuals can benefit by the use of the present invention. For example, the present invention can be used to treat any individual who is afflicted with one or more bodily conditions which are pre-cardiovascular indicators of the development of CVD. Such bodily conditions are known. Examples of such bodily conditions are high blood pressure, obesity, and Type 2 diabetes. An individual who has a pre-disposition to the development of CVD as a result of genetic makeup can benefit also by use of the present invention. Also, it is well established that an individual can engage in conduct which causes the development of a bodily condition which is not readily apparent or identifiable, but which exists, nevertheless, and leads to the development of CVD, including death without warning. Various types of such conduct are known in the art; examples are smoking, insufficient or inadequate daily exercise, and becoming obese.

Also, it has been reported that individuals who have an HbA1C of greater than 5.7% are at high risk for heart disease and stroke. It is recommended that the present invention be used with regularity by an individual whose age is about 65 and over.

In addition, an individual may intentionally or even unintentionally be exposed to disease-causing environmental conditions, for example, being exposed to radon gas or to aromatic or halogenated solvents.

An individual who does not have CVD or a bodily condition which is a pre-cardiovascular indicator of the development of CVD can benefit also by use of the present invention in a prophylactic way, that is, to deter the development of a bodily condition which is associated with the cause of CVD. An example of such an individual is one who appears to be healthy in the cardiovascular sense and who lives a life style which is recognized as being effective in reducing the risk of the development of one or more bodily conditions which lead to CVD. The use of the present invention by such an individual can be effective in delaying the onset or warding off the development of such bodily condition that tends to develop naturally as an individual ages. It is recommended that the present invention be used with regularity in a prophylactic way by a “healthy” individual who reaches the age of about 30 and thereafter.

In addition, the present invention can be used to treat effectively an individual who is not taking medication of the type which is effective in treating a bodily condition which is associated with CVD, for example, medication which controls the level of cholesterol and/or which is a blood thinner (anti-coagulant) and/or which is associated with kidney or liver damage. Nevertheless, the present invention can be used also in combination with such medications, depending on how the individual responds to treatment with only the regimen of the present invention.

In addition, the present invention can be used to treat any individual who has CVD. Such use can mitigate the adverse effects of CVD and/or deter the worsening of the condition or conditions associated with CVD.

As regards Alzheimer's disease (also referred to herein as “Alzheimer's”), it is accepted that this disease is the most common cause of the loss of mental function in individuals of age 65 and over. The disease is a brain disorder in which the individual suffers, for example, from loss of memory, language skills, and perception of time and space; such losses worsen with time. Eventually the individual is unable to care for him or herself. Although the disease exists in the main in individuals aged 65 and older, earlier-onset Alzheimer's is known also to exist in younger individuals, although rarely. As individuals grow older, the risk of developing the disease increases.

There are various explanations as to why an individual becomes afflicted with Alzheimer's. For example, it has been established that there are particular groups of individuals who are more susceptible to developing the disease (high-risk individuals) than individuals who comprise the general population. For example, research has shown that individuals who have a family history of Alzheimer's are more apt to develop the disease than individuals who do not have such family history. Another exemplary group of such high-risk individuals consists of those who have suffered brain injuries, for example, from vehicle crashes, falls, impacts to the head, and hemorrhages from burst blood vessels. Research has shown that individuals who have suffered such brain trauma often develop in later life dementia, including, for example, Alzheimer's. Still another group of such high-risk individuals are Type 2 diabetics; it has been reported that they have two to four times the risk for developing Alzheimer's and that, in incipient Alzheimer's, conditions are accelerated in Type 2 diabetics relative to the general population.

There has been a very substantial amount of research conducted on the brains of individuals who have been diagnosed with Alzheimer's and who have died. Such research has revealed, among other things, that “such” brains have characteristics which are distinctive. For example, they include proteins which are abnormally shaped, including, for example, abnormally shaped proteins which are called “plaques” (also referred to as “senior plaque deposits”) and which are formed mostly in the regions of the brain that are related to the function of memory. It has been reported that an amyloid precursor protein forms toxic plaques which cause neurons in the brains of individuals with Alzheimer's to shrink and eventually die; it is believed that this leads to some of the terribly undesirable symptoms of the disease.

An important benefit associated with the use of the present invention in connection with its applicability to CVD is that it can be effective also in postponing the development in an individual of Alzheimer's or prevent its development; this is an example of the prophylactic use of the present invention. In addition, the present invention can be used to advantage to treat an individual who is afflicted with Alzheimer's. It is believed that this is due, at least in part, to the effectiveness of the regimen of the present invention to control in the individual, particularly Type 2 diabetics, the level of blood sugar (glucose) and/or to slow the formation in the brain of senior plaque deposits.

The appropriate use of a glucose-lowering drug (G-LD) in the regimen of the present invention is effective in lowering elevated levels of glucose in the blood to near normal ranges; such control of the glucose levels has the effect of improving the quality of life of a Type 2 diabetic and giving the individual the opportunity to live a relatively long and healthy life. This is accomplished by eliminating or lessening the adverse symptoms associated with Type 2 diabetes. The use of the regimen of the present invention should be accompanied preferably by the adoption of the Type 2 diabetic of a lifestyle that is medically recommended for Type 2 diabetics, for example, eating a balanced diet that limits and spreads carbohydrate ingestion throughout the day, engaging in regular exercise, refraining from smoking, and limiting caloric intake.

The regimen of the present invention includes one or more G-LDs. Many types of G-LDs are known, examples of which include metformin, a sulfonylurea (for example, glyburide, and glipizide), thiazolidinedione and acarbose.

The daily amount of a G-LD used in the regimen of the present invention is a pharmaceutically effective amount, that is, an amount that will reduce the blood glucose level to a normal or near normal range. The particular G-LD used and amount of the drug used will depend, as explained hereinafter, on various other factors.

Speaking generally, however, metformin is recommended to be used daily in an amount within the range of about 500 to about 2,550 mg. Acarbose is generally recommended to be used daily in an amount in the range of about 50 to about 300 mg. Glyburide is generally recommended for daily use in an amount of about 1 to about 20 mg. Taking into account that there are other available glucose-lowering drugs, it is believed that the most widely used regimens will include an amount of a glucose-lowering drug or a mixture of such drugs that falls within the range of about 1 to about 3,000 mg on a daily basis.

In preferred form, the regimen of the present invention comprise a mixture of about 0.5 to about 40 mg. of glyburide and metformin in an amount of about 1000 to about 2500 mg.

A factor to consider in selecting the particular G-LD and amount to use is the age of the individual being treated with the regimen. For example, for an individual whose age is about 65 or over, it is recommended that the individual be treated with the aforementioned preferred mixture comprising glyburide and metformin in which the glyburide content is within the range of about 0.5 to about 2.5 mg.

The glucose-lowering drug can be used conveniently and effectively by oral ingestion of, for example, a tablet; accordingly, the injection into the blood stream of insulin or the like is not necessary, that is, the use of the regimen is “injection-free” in its preferred form. In special cases, injection of the drug may be necessary, such as, for example, in severe cases of the disease which require hospitalization or during pregnancy or breast feeding. The glucose-lowering drug can be administered also as a component of a composition which contains one or more other constituents of the regimen and which is in any suitable form associated with pharmaceutical compositions.

The G-LD-containing regimen of the present invention comprises one of the following NSAIDs: magnesium salicylate, naproxen, and ibuprofen, or a mixture of two or three thereof.

The Mg salicylate, is used preferably in its tetrahydrate form (hereafter for convenience “Mg salicylate” which is available commercially as efflorescent colorless crystals; they are soluble in water and alcohol. Mg salicylate is well known and is available in the U.S. over the counter for use in relieving pain and inflammation attributed to various conditions, for example, arthritis, bursitis, and tendonitis.

For use in the regimen of the present invention, an NSAID can be administered orally, for example, as a tablet, gelcap, or caplet, or it can be administered as a component of a composition which contains one or more other ingredients of the regimen and which is in any suitable form associated with pharmaceutical compositions that are taken orally.

The daily amount of Mg salicylate used in the regimen of the present invention is a pharmaceutically effective amount, but an amount not greater than about 265 mg, for example, an amount within the range of about 130 to about 265 mg on a daily basis. The Mg salicylate can be ingested once daily or more than once, for example, twice a day, and preferably at breakfast or with another meal. As explained hereinafter, the particular amount used will depend on various factors.

The regimen of the present invention can include also naproxen or ibuprofen. Each is compatible with each other and with Mg salicylate and with other constituents of the regimen. Each can be administered orally, for example, as a tablet, gelcap, or caplet, or it can be administered as a component of a composition which contains one or more other ingredients of the regimen and which is in any suitable form associated with pharmaceutical compositions that are taken orally.

Naproxen comprises the molecule C₁₂H₁₄O_{3;} it is available as a sodium salt. Naproxen is a popularly used drug that is effective in alleviating pain, reducing fever, and inflammation and stiffness caused by many types of conditions; for example, various types of arthritis and bursitis. Naproxen functions by reducing hormones that cause bodily inflammation and pain. It is available in immediate-release and delayed-release form. One form of naproxen is sold under the trademark NAPROSYN®. Naproxen can be ingested once daily or more than once, for example, twice a day in an amount within the range of about 30 to about 220 mg on a daily basis.

As to the NSAID ibuprofen, it is chemically defined as isobutylphenylpropanoic acid. An exemplary form of ibuprofen is sold under the trademark ADVIL®. Ibuprofen can be ingested once or more than once daily in a pharmaceutically effect amount, for example, about 40 to about 200 mg.

Another constituent for use in the G-LD-containing regimen of the present invention is acetaminophen; it is an organic compound that is a popularly used drug which is effective in reducing fever, headaches, and other aches and pains. It is an analgesic, but it is not an NSAID because it has no anti-inflammatory properties. For pain relief, it functions by inhibiting synthesis of prostaglandins in the central nervous system and for reducing fever, it functions by having an effect on the temperature-regulating center of the brain.

Acetaminophen can be used in forms such as described above for NSAIDS. The daily amount of acetaminophen used in the present invention is a pharmaceutically effective amount, for example, about 400 to about 2500 mg; it can be ingested once daily or more than once, for example, twice a day.

Another aspect of the present invention is the provision of a regimen (hereafter the “CVD/Alz regimen) for use in deterring the development in an individual of a cardiovascular disease or of Alzheimer's and/or for treating individuals who are affected with such diseases. Examples of embodiments of this regimen include the ingestion by an individual on a daily basis of:

(A) at least one non-steroidal anti-inflammatory drug (NSAID); and

(B) acetaminophen.

Aforementioned ingredients (A) and (B) immediately above are described hereinbefore in connection with the G-LD-containing regimen of the present invention and can be used in the daily amounts described hereinbefore. The CVD/Alz regimen can be used in applications in which a medical determination has been made that it is not necessary for the individual to be treated with a G-LD.

A particular individual may have a health condition or disease which is being treated with one or more drugs pursuant to medical advice. The regimens of the present invention can include also the use of a prescribed medication which is compatible with other drugs of the regimens. For example, the regimen can include drugs which are designed to treat hypertension, for example, enalapril maleate (an ACE inhibitor), toprol/metoprolol tartrate and other beta blockers. An individual who has a condition involving benign prostate enlargement (BHP) can include in the regimen, for example, FLOWMAX, HYTRIN, or CARDURA (doxazosinmesylate, an alpha-1-blocker); the last mentioned drug has dual functions in that it increases urinary flow and reduces high blood pressure. The use of the regimens of the invention can be accompanied also by treatment of glaucoma with one or more appropriate drugs, for example, an eye-drop solution of timolol, brimonidine tartrate, or xalatan. Two or more of “glaucoma” drugs can be used. It should be understood that the aforementioned are exemplary and that other medications can be used with the present regimens as may be desirable or necessary for the treatment of an ailment in the individual.

The amount of such other constituent to include in the regimens or that accompanies the use of the regimens is a pharmaceutically effective amount, as determined by those skilled in the art.

As mentioned above, the particular amount of drug used in a regimen depends on a number of factors; consider the following as general guidelines. For example, when using the regimen in a prophylactic way, the drugs can be used in amounts toward the lower end of the amount range, particularly in early stage Type 2 diabetes. In situations in which the individual has one or more bodily conditions or engages in conduct that are pre-cardiovascular indicators of the development of CVD, the drugs can be used in amounts within the middle of the amount range. Similarly, with individuals who are considered at high risk to develop Alzheimer's, the drugs can be used in amounts within the middle of the amount range. For those who have already developed CVD or Type 2 diabetes or Alzheimer's, the drugs can be used in amounts toward the higher end of the amount range at age 55 to 70. Similarly, the drugs can be used in amounts toward the higher end of the amount range with older individuals who are afflicted with CVD or Type 2 diabetes or Alzheimer's. Typically, individuals who have a relatively high body mass index (BMI), that is, a BMI, which characterizes the individual as overweight or obese, will also benefit by use of the present invention. As mentioned above, the aforementioned statements respecting “amounts of drugs” should be considered general guidelines. For any particular individual, adjustments in the amounts of drugs used can be made as test results show that adjustment is warranted.

The regimens of the present invention can be in any suitable form that is used typically in medical applications. For example, each constituent comprising a regimen can be contained in an individual package, including, for example, a package which is associated with one or more packages containing other of the constituents comprising the regimens.

### EXAMPLES

There follow examples which are illustrative of the practice of the present invention.

There are set forth in the Table below the results of tests of blood and urine collectable from a male individual (test subject) who was born in 1927 and who was diagnosed with Type 2 diabetes in 1984. The individual has a family history of cardiovascular disease (CVD); the death of each parent was caused by a stroke (mother at age 62 and father at age 86). The individual has not suffered any affliction associated with CVD during his entire lifetime; he has never used statins. The BMI of the individual at ages 70-85 ranged from 30 to 33 lbs.

The results reported below include test results which were carried out about 90 days after he began use of the daily regimen which is described below and involved oral ingestion of each of the ingredients of the regimen.
(1) 1,500 mg of metformin taken in the form of 500 mg tabs usually at 3 main meals.
(2) 2.5 mg of glyburide;
(3) 100 mg of ibuprofen in the form of a tablet in the morning;
(4) 1,000 mg of acetaminophen taken in the form of a tablet every 8 hours.
   In addition to the ingredients of the regimen, he ingested also during the approximate 90-day period hypertension pills and glaucoma eye drops.

Within 90 days after use of the regimen, the patient at age 88 visited his doctor who issued the following report after evaluating laboratory rest results based on the patient's blood sample.
(1) All of the patient's lab work was considered normal or near normal. This included blood counts, blood sugar, liver and kidney testing.
(2) Cholesterol Studies: Normal
   Cholesterol: 139 Goal: (<200 mg/dL)
   HDL: 32 Goal: (>40 mg/dL)
   LDL: 83 Goal: (<130 mg/dL)
   Non-HDL: 107 Goal: (<130 mg/dL)
   Triglyceride: 120 Goal: (<150 mg/dL)
(3) Blood Glucose Studies: Normal.
   Blood Glucose: 89 Goal: (70-100 mg/dL Fasting)
   HGBA1C: 4.7 Goal: (4.2-5.8%)

In another embodiment of the invention, the above regimen was changed by reducing the amount of the glyburide constituent of 1.25 mg. Within 90 days after use of the “changed” regimen, the patient visited his doctor who issued the following report after evaluating laboratory test results based on the patient's blood sample.
(1-A) All of the patient's lab work was considered normal or near normal. This included blood counts, blood sugar, liver and kidney testing.
(2-A) Blood glucose studies: near normal;
   HGBA1C: 5.9

1. A method for treating an individual comprising the use by the individual of a regimen which includes the ingestion daily by the individual of a pharmaceutically effective amount of:
(A) one or more glucose-lowering drugs (G-LDs) which function to lower the amount of glucose in the blood;
(B) at least one non-steroidal anti-inflammatory drug (NSAID) selected from the group consisting of (1) magnesium salicylate tetrahydrate (hereafter “Mg salicylate”); (2) naproxen; and (3) ibuprofen; and
(C) acetaminophen.

2. A method according to claim 1 in which the individual has Type 2 diabetes.

3. A method according to claim 1 in which the individual is treated with the regimen in order to prevent or to delay the onset of Type 2 diabetes in the individual.

4. A method according to claim 1 in which the individual lives a life style which reduces the severity of undesirable symptoms associated with Type 2 diabetes.

5. A method according to claim 1 in which the G-LD is selected from the group consisting of one or more of metformin, glyburide, glipizide or other sulfonyl urea, thiazolidinedione, and acarbose.

6. A method according to claim 1 in which the regimen includes an amount of a glucose-lowering drug or a mixture of such drugs within the range of about 1 to about 3,000 mg on a daily basis.

7. A method according to claim 1 in which the amount of Mg salicylate is not greater than about 265 mg.

8. A method according to claim 7 in which the amount of Mg salicylate is within the range of about 130 to about 265 mg.

9. A method according to claim 1 in which the amount of naproxen is within the range of about 30 to about 220 mg.

10. A method according to claim 1 in which the amount of ibuprofen is within the range of about 40 to about 200 mg.

11. A method according to claim 1 in which the amount of acetaminophen is within the range of about 400 to about 2500 mg.

12. A method according to claim 1 in which the individual has Type 2 diabetes and whose age is at least about 65 years and in which the regiment consists essentially of a mixture of about 0.5 to about 2.5 mg of glyburide and about 1000 to about 2500 of metformin.

13. A method according to claim 1 in which the regiment comprises about 1000 to about 2500 mg of metformin, about 1 to about 20 mg of glyburide, about 40 to about 200 mg of ibuprofen, and about 400 to about 2500 mg of acetaminophen.

14. A method according to claim 13 in which the individual has Type 2 diabetes.

15. A method for use in deterring the development in an individual of a cardiovascular disease or of Alzheimer's and/or for treating an individual who is effected with one or more of such diseases comprising the ingestion by the individual on a daily basis of:
(A) at least one non-steroidal anti-inflammatory drug (NSAID) selected from the group consisting of magnesium salicylate tetrahydrate (hereafter Mg salicylate, naproxen, and ibuprofen; and
(B) acetaminophen.

16. A method according to claim 15 in which the regimen comprises either about 130 to about 265 mg of Mg salicylate or about 30 to about 220 mg of naproxen or about 40 to about 200 mg of ibuprofen and also about 400 to about 2500 mg of acetaminophen.

17. A regimen comprising multi-ingredients which are associated with each other for use in treating on a daily basis an individual in order to prevent or to delay the onset of Type 2 diabetes in which the regimen comprises in pharmaceutically effective amounts:
(A) one or more glucose-lowering drugs (G-LDs) which function to lower the amount of glucose in the blood;
(B) at least one non-steroidal anti-inflammatory drug (NSAID) selected from the group consisting of (1) magnesium salicylate tetrahydrate (hereafter “Mg salicylate”); (2) naproxen; and (3) ibuprofen; and
(C) acetaminophen.

18. A regimen according to claim 17 in which the G-LD is selected from the group consisting of one or more of metformin, glyburide, glipizide or other sulfonyl urea, thiazolidinedione and acarbose.

19. A regimen according to claim 18 in which the regimen includes an amount of a glucose-lowering drug or a mixture of such drugs within the range of about 1 to about 3,000 mg and also acetaminophen in an amount of about 400 to about 2500 mg and in which the multi-ingredients are associated with each other in a convenient to use dosage form.

20. A regimen according to claim 17 in which the regimen includes about 0.5 to about 25 mg of glyburide and about 1000 to about 2500 mg of metformin.
